(19) United States
(12) Patent Application Publication
(10) Pub. No.: US 2005/0176807 A1
(43) Pub. Date: Aug. 11, 2005

(54) Composition and method for use in cartilage affecting conditions

(76) Inventors: Kim Gene Friesen, Topeka, KS (US); Phillip W. Toll, Valley Falls, KS (US)

Correspondence Address: Harness Dickey & Pierce P.L.C.;James E Davis, 7700 Bonhomme Suite 400, St. Louis, MO, 63105, US

(21) Appl. No.: 10/774,951

(22) Filed: Feb. 9, 2004

(51) Int. Cl. A61K 31/401; A61K 31/198

(52) U.S. Cl. 514/423; 514/561

(57) ABSTRACT

A method for increasing the flexibility of cartilage in an animal in need of such increase which comprises administering to said mammal a cartilage flexibility enhancing effective amount of at least one of glycine and proline.

### BACKGROUND OF THE INVENTION

Virtually all joints have cartilage. Cartilage is important in the body of animals for providing flexibility, compressability under pressure, cushion, and tensile strength range and smoothness of movement within joints. Healthy well developed cartilage is relatively resistant to deterioration over time. Poorly developed cartilage is more susceptible to damage that leads to disease. Examples of joints having cartilage include fingers and toes, neck, knee, hip, shoulder and the like. Animals can suffer from a number of conditions where cartilage is negatively affected thereby bringing about a reduction in the joint's flexibility, strength and often times resulting in a generalized inflammation of the joint and/or tissue surrounding the joints. Such animal then has significant loss of joint function and experiences pain. These conditions include arthritis, osteo and rheumatoid, osteochondrosis, degenerative joint disease, synovitis, bacterial purulent arthritis, osteoarthropathia and psoriatica among others.

We have now found a method and composition for preventing loss of flexibility and/or strength in those animals in need of said assistance. These animals can already have classical symptoms of the condition(s) or can be susceptible of such condition(s), the latter, for example, being a large breed dog having hip dysplasia problems which can bring about arthritis or similar conditions. Such assistance can even be given to animals in no apparent immediate need of such assistance but wherein growth of cartilage occurs as in the younger years or approaching an age where such conditions are relatively commonplace, for example “old age”.

The assistance is provided by the use of certain quantities of glycine and/or proline, administered in a systemic manner, such as orally, in a food, liquid or dosage unit form. The data in the specification shows that cartilage flexibility and strength as measured by compressiblity or initial fracture are substantially increased using the invention. Both flexibility and strength enhance joint health and make the joint less susceptible to physical damage and cartilage degradation conditions such as arthritis, both in a preventive and treatment mode.

### SUMMARY OF THE INVENTION

In accordance with the invention, there is a method for increasing the flexibility of cartilage in an animal in need of such increase which comprises administering to said animal a cartilage flexibility enhancing effective amount of at least one of glycine and proline.

A further aspect of the invention is a method for increasing the strength of cartilage in an animal in need of such increase which comprises administering to said animal a cartilage strength enhancing effective amount of at least one of glycine and proline.

Another aspect of the invention is a method for preventing degradation of cartilage tissue in an animal in need of said prevention which comprises administering to said animal a cartilage degradation prevention effective amount of at least one of glycine and proline.

An additional aspect of the invention is a composition suitable for systemic administration to an animal comprising a cartilage flexibility or cartilage strength enhancing effective amount of at least one of glycine and proline in association with a carrier.

There are many other aspects of the invention are disclosed throughout this specification.

### DETAILED DESCRIPTION OF THE INVENTION

Animal as used throughout the specification includes human, dog, cat, horse, goat, sheep, swine, cattle, birds including turkeys and chickens, and the like. Preferred are humans, dogs, cats, horses and swine.

Cartilage affecting conditions are those which are particularly managed by the administration of the glycine and/or proline. Illustrative examples of such conditions include osteoarthritis, rheumatoid arthritis, osteochondrosis, degenerative joint disease, synovitis, bacterial purulent arthritis, osteoarthropathia, and psoriatica.

The active material(s) of the invention can be administered in any systemic manner.

The glycine and/or proline can be administered to the animal, preferably one in need of such administration in any one of many ways, such as oral, parenteral, and the like, although oral is preferred. The glycine and/or proline can be administered in a wet or dry diet, either incorporated therein or on the surface of any diet component, such as, by spraying or precipitation thereon. It can be present in the nutritional diet per se or in a snack, supplement or a treat. It can also be present in the liquid portion of the diet such as water or another fluid. The glycine and/or proline can be administered as a powder, solid or as a liquid including a gel. If desired the glycine and/or proline can be orally administered in a pharmaceutical dosage form such as a capsule, tablet, caplet, syringe, and the like. Within the dosage form the glycine and/or proline can be present as a powder or a liquid such as a gel. Any of the usual pharmaceutical carriers can be employed such as water, glucose, sucrose and the like together with the active(s). Although exemplified together, the glycine and proline when used together can be administered separately, that is one in a diet and one in a liquid or a unit dose form, for example. Generally, when administered together, they should be administered at least concomitantly, preferably in the same carrier. When administered in a food, the glycine and/or proline can be administered as a compound, within the normal food constituents or a combination of the two.

With respect to prevention of joint damage from arthritis, particularly osteo, or other noted conditions, a particular target group of pets, especially canines, and felines are those that would be in need of such preventative care as opposed to the general population. For example, pets, particularly large breed canines such as labrador retriever, rottweiler, german shepherd and the like are more susceptible to arthritis as demonstrated by its greater occurence in these pets. Additionally, pets above the age of six (6) years, particularly dogs and cats, have a significantly greater occurrence of arthritis, particularly osteo arthritis. Other examples of pets susceptible to the development of arthritis include horses. The compounds can be additionally useful in treating animals, especially canines and felines with arthritis, particularly osteo and other noted conditions.

The quantity of glycine and/or proline which should be employed can vary substantially. All wt % are based on dry matter basis of a daily diet sufficient to satisfy the nutrition needs of the animal. A minimum amount of proline is about 1.45 wt %, preferably about 1.6 wt % and more preferably about 2.0 wt %. A minimum amount of glycine is about 1.25 wt %, preferably about 1.4 wt % and more preferably about 1.6 wt %. For example, a specific amount can be employed in the usual nutrient food ration on a daily basis or the same daily quantity can be provided to the animal in a treat or supplement on a daily basis. Additionally, a combination of these methods or any other dosing means can be employed as long as the effective quantity of the active constituent is provided. Maximum quantities are any amount effective with little (acceptable level) or no toxicity. Examples of such quantities for glycine and proline, each include not more than about 5, 4, or 3 wt % on the same basis as for the minimums.

As aforementioned, the glycine and/or proline can be in any food provided to the pet. Examples of such foods are regular diets providing all of the animal's nutrients, treats, supplements and the like. The actives can be provided in liquids or in pharmaceutical dosage forms such as capsules, tablets, pills, liquids or even parenterally administered through syringe. The most important aspect is that the pet be provided an effective amount of active to provide the positive cartilage effect. The preferred route of aministration is oral and incorporated with a food. Foods are generally classified in the pet food industry as “wet” or “dry”. A wet food has a relatively high amount of water and is usually present in a can or a container wherein air is substantially or totally excluded. Examples of such foods are “chunk and gravy”, individual solid particles in the presence of a liquid gravy or a loaf type material which generally takes the shape of the receptacle. The dry food is generally a baked or preferably extruded material, the latter then cut into individual shaped portions, usually known as kibbles. The actives are readily incorporated into a wet food through conventional means.

With respect to pet food such as for dog and cat the wt % ranges on a dry matter basis of protein, fat and carbohydrate for a dog is: protein=15-55 wt %; fat=5-40 wt %; and carbohydrate=10-50 wt % and for a cat is: protein=15-55 wt %; fat=5-40 wt %; and carbohydrate=10-50 wt %.

Below are examples. These examples are illustrative exemplification of the scope of the invention.

The Instron techniques used are as disclosed in:

1. Otremba, M. M., M. E. Dikeman, G. A. Milliken, S. L. Stroda, J. A. Unruh, and E. Chambers IV. 1999. Interrealtionships among evaluations of beef longissimus and semitendinosis muscle tenderness by Warner-Bratzler shear force, a descriptive-texture profile sensory panel and a descriptive attribute sensory panel. J. Anim. Sci. 77:865-873;

2. Wheeler T. L., S. D. Shackelford, and M. Koohmaraie. 1998. Cooking and palatability traits of beef longissimus steaks cooked with a belt grill or an open hearth electric broiler. J. Anim. Sci. 76:2805-2810; and

3. Wheeler T. L., S. D. Shackelford, and m. Koohmaraie. 2002. Technical note: Sampling methodology for relating sarcomere length, collagen concentration, and the extent of postmortem proteolysis to beef and pork longissimus tenderness. J. Anim. Sci. 80:982-987.

Growing pigs (80) were used as a test model in the examples. The pigs initially were about 35 kg. Each pig was individually housed in 5.2 ft² pens with ad libitum access to food and water. The pigs were fed test foods for a period of 60 days to an approximate final weight of 130 kg.

At the point of meat fabrication, the right stifle joint was harvested from each pig, and the articular cartilage from the patella bone was removed. The cartilage weight and thickness (average of three measures) was determined prior to mechanical analyses. These measures were used to determine the compression distance (50% of the average cartilage thickness) by Instron analyses of flexibility and Warner-Brazler shear force.

Warner-Brazler Shear Force was determined using an Instron Universal testing machine (model 4201, Instron Corporation, Canton, Mass.). A 50 kg compression load cell with a crosshead speed of 250 mm/min. was used. The machine was assembled with a flat retractable blade which was lowered onto a sample placed on a stationary anvil. The press exerted a force that severs the sample on the stationary anvil. The procedure measures the force (kg) required to initially fracture the cartilage sample.

Cartilage flexibility was measured in the following manner. Compression was measured by placing the cartilage sample on a stationary plate, while the press lowered a plate onto the top of the sample. The press was lowered to compress the sample 50% of the average thickness. The force required to compress the sample 50% was the measure of flexibility, and the lower the force, the more flexible the sample.

Units of measure for shear force data:
a. Shear force (both peak and total force) is kilograms
b. The energy measure is Newtons.

### EXAMPLES 1 AND 2

TABLE 1
Composition of Experimental Foods

| | Control | Example 1 | Example 2 |
|---|---|---|---|
| Corn | 71.00 | 71.00 | 78.00 |
| Soybean meal | 18.70 | 18.70 | 1.39 |
| Corn Starch | 3.78 | 2.23 | 3.00 |
| Ch White Grease | 3.00 | 3.00 | 0.50 |
| Dical | 1.97 | 1.98 | 1.20 |
| Limestone | 0.62 | 0.77 | 0.26 |
| Salt | 0.43 | 0.55 | 0.29 |
| L-lysine | 0.15 | 0.15 | |
| Vitamin premix | 0.10 | 0.10 | 0.10 |
| Choline | 0.10 | 0.10 | 0.10 |
| TM premix | 0.10 | 0.10 | 0.10 |
| Mn sulfate | | | 0.02 |
| Tryptophan | | | 0.05 |
| Poultry Meal | | | 12.00 |
| Gelatin | | | 3.00 |
| Proline | | 0.48 | |
| Glycine | | 0.80 | |
| DL-methionine | 0.04 | 0.04 | |
| Total | 100 | 100 | 100 |
| 100% DM basis | | | |
| ME | 3604 | 3604 | 3608 |
| Ca | 0.86 | 0.85 | 0.85 |
| P | 0.74 | 0.74 | 0.74 |
| Na | 0.22 | 0.22 | 0.22 |
| Lys | 0.97 | 0.97 | 0.96 |
| TSAA | 0.58 | 0.58 | 0.59 |
| Trp | 0.20 | 0.20 | 0.20 |
| Thr | 0.66 | 0.66 | 0.73 |
| Iso | 0.65 | 0.65 | 0.63 |
| Pro | 1.12 | 1.69 | 1.74 |
| Gly | 0.78 | 1.69 | 2.10 |

TABLE 2
Analytical analyses of experimental foods - lot 1

| | Control | Example 1 | Example 2 |
|---|---|---|---|
| Crude protein | 17.32 | 19.29 | 20.46 |
| Fat | 7.76 | 8.04 | 6.54 |
| Fiber | 2.05 | 2.26 | 1.61 |
| Proline | 1.13 | 1.62 | 1.69 |
| Glycine | 0.65 | 1.40 | 1.70 |
| Hydroxyproline | 0.05 | 0.12 | 0.63 |

TABLE 3
Analytical analyses of experimental foods - lot 2

| | Control | Example 1 | Example 2 |
|---|---|---|---|
| Crude protein | 17.38 | 17.97 | 20.58 |
| Fat | 6.83 | 7.42 | 6.28 |
| Fiber | 2.91 | 2.35 | 1.71 |
| Proline | 1.10 | 1.52 | 1.70 |
| Glycine | 0.66 | 1.31 | 1.80 |
| Hydroxyproline | 0.05 | 0.05 | 0.66 |

TABLE 4
Effect of nutrition on cartilage flexibility

| Item | Control | Example 1 | Example 2 |
|---|---|---|---|
| Peak force, kg | 10.47 | 4.47 | 4.85 |
| Peak energy, Neutons | 1.54 | 0.50 | 0.44 |

The above tables provide the composition of the experimental foods as well as the control, see Table 1. Tables 2 and 3 provide analytical analyses of two lots of the control and experimental foods. Table 4 provide the results of the comparison testing. The results clearly show that statistically significantly less energy is needed to compress the cartilage to the 50% level utilizing higher levels of glycine and proline than the control which has less glycine and proline than either of the two examples.

The pig cartilage is now tested for its breaking strength, that is the amount of force it takes to bring about the initial notching or begin shearing of cartilage from bone. The greater the amount of force, the stronger is the bonding between cartilage and the bone and the more resistant cartilage is to arthritic or arthritic type symptomatology.

### EXAMPLES 3, 4 AND 5

TABLE 5
Composition of Experimental Foods

| | Control | Example 3 | Example 4 | Example 5 |
|---|---|---|---|---|
| Corn | 71.00 | 78.50 | 71.00 | 78.00 |
| Soybean meal | 18.70 | 3.35 | 18.70 | 1.39 |
| Corn Starch | 3.78 | 3.00 | 0.71 | 3.00 |
| Ch White Grease | 3.00 | 1.00 | 3.00 | 0.50 |
| Dical | 1.97 | 1.13 | 1.98 | 1.20 |
| Limestone | 0.62 | 0.28 | 0.76 | 0.26 |
| Salt | 0.43 | 0.31 | 0.53 | 0.29 |
| L-lysine | 0.15 | 0.08 | | |
| Vitamin premix | 0.10 | 0.10 | 0.10 | 0.10 |
| Choline | 0.10 | 0.10 | 0.10 | 0.10 |
| TM premix | 0.10 | 0.10 | 0.10 | 0.10 |
| Mn sulfate | | 0.02 | | 0.02 |
| Tryptophan | | 0.03 | | 0.05 |
| Poultry Meal | | 12.00 | | 12.00 |
| Gelatin | | | 3.00 | 3.00 |
| DL-methionine | 0.04 | | 0.01 | |
| Total | 100 | 100 | 100 | 100 |
| 100% DM basis | | | | |
| ME | 3604 | 3634 | 3604 | 3608 |
| Ca | 0.86 | 0.85 | 0.85 | 0.85 |
| P | 0.74 | 0.74 | 0.74 | 0.74 |
| Na | 0.22 | 0.22 | 0.22 | 0.22 |
| Lys | 0.97 | 0.96 | 0.97 | 0.96 |
| TSAA | 0.58 | 0.60 | 0.58 | 0.59 |
| Trp | 0.20 | 0.20 | 0.20 | 0.20 |
| Thr | 0.66 | 0.70 | 0.72 | 0.73 |
| Iso | 0.65 | 0.65 | 0.69 | 0.63 |
| Pro | 1.12 | 1.15 | 1.48 | 1.74 |
| Gly | 0.78 | 1.15 | 1.48 | 2.10 |

TABLE 6
Analytical analyses of experimental foods - lot 1

| | Control | Example 3 | Example 4 | Example 5 |
|---|---|---|---|---|
| Crude protein, % | 17.32 | 18.34 | 19.20 | 20.46 |
| Fat, % | 7.76 | 7.58 | 6.97 | 6.54 |
| Fiber, % | 2.05 | 1.73 | 2.35 | 1.61 |
| Proline, % | 1.13 | 1.34 | 1.50 | 1.69 |
| Glycine, % | −6.5 | 1.05 | 1.17 | 1.70 |
| Hydroxyproline, % | −0.5 | .25 | .36 | .63 |
| Manganese, ppm | 46.42 | 81.23 | 53.18 | 93.34 |

TABLE 7
Analytical analyses of experimental foods - lot 2

| | Control | Example 3 | Example 4 | Example 5 |
|---|---|---|---|---|
| Crude protein, % | 17.38 | 18.43 | 19.30 | 20.38 |
| Fat, % | 6.83 | 7.87 | 7.57 | 6.28 |
| Fiber, % | 2.91 | 1.62 | 2.25 | 1.71 |
| Proline, % | 1.10 | 1.37 | 1.44 | 1.70 |
| Glycine, % | .66 | 1.19 | 1.19 | 1.80 |
| Hydroxyproline, % | .05 | .34 | .36 | 0.66 |
| Manganese, ppm | 41.84 | 96.81 | 43.54 | 102.11 |

TABLE 8
Effect of nutrition on cartilage breaking strength

| Item | Control | Example 3 | Example 4 | Example 5 |
|---|---|---|---|---|
| Peak force, kg | 67.46 | 71.79 | 76.83 | 112.32 |
| Peak energy, Neutons | 39.42 | 40.33 | 48.62 | 55.76 |

As observed from the results, the higher quantities of glycine and proline, see Example 5, brings about the strengthening of the cartilage. Therefore, the joint including the cartilage is more resistant to initiation of exacerbation of arthritis or arthritis-like symptomatology. It is noted that the results of the experiments can apply to the other cartilage affecting conditions previously mentioned conditions as well as arthritis.

1. A method for increasing the flexibility of cartilage in an animal in need of such increase which comprises administering to said animal a cartilage flexibility enhancing effective amount of at least one of glycine and proline.

2. The method in accordance with claim 1 wherein the animal has a condition or needs prevention of a condition selected from the group consisting of osteoarthritis, rheumatoid arthritis, osteochondorosis, degenerative joint disease, synovitis, bacterial purulent arthritis, osteoarthropathia and psoriatica.

3. The method in accordance with claim 1 wherein a cartilage flexibility enhancing amount of glycine is employed.

4. The method in accordance with claim 3 wherein a minimum of at least about 1.25 wt % of glycine is employed based on dry matter basis of diet.

5. The method in accordance with claim 1 wherein a cartilage flexibility enhancing amount of proline is employed.

6. The method in accordance with claim 5 wherein a minimum of at least about 1.45 wt % of proline is employed based on dry matter basis of diet.

7. The method in accordance with claim 1 wherein a cartilage flexibility amount of glycine and proline is employed.

8. The method in accordance with claim 7 wherein the animal is a dog or cat.

9. A method for increasing the strength of a cartilage in an animal in need of such increase which comprise administering to said animal a cartilage strength enhancing effective amount of at least one of glycine and proline.

10. The method in accordance with claim 9 wherein the animal has a condition or needs prevention of a condition selected from the group consisting of osteoarthritis, rheumatoid arthritis, osteochondorosis, degenerative joint disease, synovitis, bacterial purulent arthritis, osteoarthropathia and psoriatica.

11. The method in accordance with claim 9 wherein a cartilage strength enhancing amount of glycine is employed.

12. The method in accordance with claim 11 wherein a minimum of at least about 1.25 wt % of glycine is employed based on dry matter basis of diet.

13. The method in accordance with claim 9 wherein a cartilage strength enhancing amount of proline is employed.

14. The method in accordance with claim 13 wherein a minimum of at least about 1.45 wt % of proline is employed based on dry matter basis of diet.

15. The method in accordance with claim 9 wherein a cartilage strength enhancing amount of glycine and proline is employed.

16. The method in accordance with claim 15 wherein the animal is a dog or cat.

17. A method for preventing degradation of cartilage tissue in an animal in need of said prevention which comprises administering to said animal a cartilage degradation effective amount of at least one of glycine and proline.

18. The method in accordance with claim 17 wherein the animal has a condition or needs prevention of a condition selected from the group consisting of osteoarthritis, rheumatoid arthritis, osteochondorosis, degenerative joint disease, synovitis, bacterial purulent arthritis, osteoarthropathia and psoriatica.

19. The method in accordance with claim 17 wherein a cartilage degradation prevention enhancing amount of glycine is employed.

20. The method in accordance with claim 19 wherein a minimum of at least about 1.25 wt % of glycine is employed based on dry matter basis of diet.

21. The method in accordance with claim 17 wherein a cartilage degradation prevention enhancing amount of proline is employed.

22. The method in accordance with claim 21 wherein a minimum of at least about 1.45 wt % of proline is employed based on dry matter basis of diet.

23. The method in accordance with claim 17 wherein a cartilage degradation preventing amount of glycine and proline is employed.

24. The method in accordance with claim 23 wherein the animal is a dog or cat.

25. A composition suitable for systemic administration to an animal comprising a cartilage flexibility or cartilage strength enhancing effective amount of at least one of glycine and proline in association with a carrier.

26. The composition in accordance with claim 25 wherein the systemic administration is oral.

27. The composition in accordance with claim 25 wherein the carrier is a dosage unit form, a food or a liquid.

28. The composition in accordance with claim 27 wherein the carrier is a food.

29. The composition in accordance with claim 26 wherein an effective amount of glycine is present.

30. The composition in accordance with claim 29 wherein at least 1.25 wt % of glycine is present based on dry matter basis of diet.

31. The composition in accordance with claim 26 wherein an effective amount of proline is present.

32. The composition in accordance with claim 31 wherein at least 1.45 wt % of proline is present based on dry matter basis of diet.

33. The composition in accordance with claim 26 wherein a combination glycine and proline are employed in at least minimum quantities.
